(19) United States
(12) Patent Application Publication
(10) Pub. No.: US 2021/0016080 A1
(43) Pub. Date: Jan. 21, 2021

(54) ELECTRODE CONTACT MONITORING

(71) Applicant: Bio-Medical Research Limited, Galway (IE)

(72) Inventor: Conor MINOGUE, Galway (IE)

(21) Appl. No.: 16/982,478

(22) PCT Filed: Mar. 29, 2019
(86) PCT No.: PCT/EP2019/058113

§ 371 Date: Sep. 18, 2020

(30) Foreign Application Priority Data
Mar. 29, 2018 (GB) 1805282.9

(51) Int. Cl. A61N 1/04; A61N 1/36

(52) U.S. Cl. CPC A61N 1/0484; A61N 2001/083; A61N 1/3603

(57) ABSTRACT

A system for assessing the quality of electrical contact in transcutaneous electrical stimulation includes an array having at least three electrodes (A, B, C) and at least two electrode pairings (AB, BC) of the array have a common electrode (B). A controller controls flow of current pulses between different electrode pairings (AB, AC, BC) of the array. A measurement device measures at least one voltage across each of the at least two electrode pairings (AB, BC) of the array during a stimulation pulse in response to a constant current pulse.

### FIELD OF THE INVENTION

The invention relates to a system and method for assessing the quality of electrical contact in transcutaneous electrical stimulation.

### BACKGROUND OF THE INVENTION

In transcutaneous electrical stimulation it is important to achieve a good quality electrical contact with the skin such that the electrical signal is transferred across the skin and into the underlying tissues while avoiding damage to the skin and minimizing any pain or discomfort due to stimulation of pain receptors. Skin electrodes are typically designed to extend over an area of skin ranging between 5 and 200 cm². Passing an electric current through the skin involves a transduction between electron current flow in the wires and metal electrodes of the stimulator system and ionic current flow in the body. This transduction takes place partly through electrolysis and therefore an electrolyte is required at the interface between the metal (or other conductive material) electrode and the skin. It is usually desirable in transcutaneous stimulation that the current density be minimised since this reduces power dissipation per unit area of skin and also reduces the likelihood of stimulating pain receptors in the skin. Normally therefore the electrolyte needs to extend over the full area of the electrode to ensure that the current density into the skin is uniform over the contact surface area. It is also important that the full available area of the electrode makes contact with the skin. If the effective electrode area is reduced, for example due to partial lifting of the electrode from the skin, then the contact area is reduced. When a constant current controlled generator is used, this means the current density in the remaining contact area is increased. This may cause skin irritation, discomfort or pain. The same applies if the electrolyte is distributed unevenly over the area of surface contact, or if the skin is partially covered by grease or dirt. This is in contrast to biophysical signal monitoring electrodes, such as electrocardiogram (ECG), where negligible current is transferred. The principle concern with such electrodes is achieving a low contact resistance and especially having similar contact resistances at each electrode to suppress common mode noise. If an ECG electrode is making incomplete contact it does not matter provided the contact resistance is low and comparable to the other electrodes. In electrical stimulation it is never acceptable that an electrode should peel to the extent that the contact area is significantly reduced.

While electrode area is important in reducing current density, the presence of an adequate electrolyte is critical to ensuring that the current is coupled across the skin in the least damaging fashion. The bulk conductivity of the electrolyte, as well as the thickness of the electrolyte layer, determine the overall sheet resistance of the interface between electrode and skin.

It is desirable therefore to have a mechanism to assess the quality of the electrical connection and in particular to estimate the area of contact.

In U.S. Pat. No. 9,474,898 B2 a solution is proposed to this problem for a series combination of two electrodes where the impedance measured during the stimulation session is divided by the baseline impedance measured at the start of the session. If this impedance ratio increases beyond an area dependent predefined value then it is assumed that the area of contact of one of the electrodes has reduced beyond that amount.

This approach has several limitations, not least that it cannot identify which of the two electrodes has become dislodged because they are in series. It could be that both of the electrodes are partially dislodged. Also, if the electrical connection at baseline is poor for some reason, for example due to the presence of skin contamination with skin cream, or due to dried out hydrogel, then the impedance ratio is flawed to begin with and the result cannot be relied upon.

Niemi (U.S. Pat. No. 4,088,141) describes a circuit for monitoring the resistance of an electrode for transcutaneous stimulation. Despite showing the waveform which occurs in response to a current pulse, it is stated that only the initial step voltage V1 is required to assess the electrode quality. Col 3 lines 55 to 68. However, this does not allow estimation of the capacitive element which is dependent on area of contact. See (Vargas Luna, Krenn et al. 2015)

Welch Allen (WO2014/047044A1) describes a system for assessing ECG electrode contact quality by applying an AC test current through a pair of electrodes and measuring a voltage on a third electrode. This approach simply measures resistance using Ohms law at a point in time and does not allow information on electrode capacitance to be assessed.

Draeger (WO 2014/021883A1) also describes using pairs of electrodes to measure resistance and resolving between between pairs to estimate the resistance of each electrode. This approach simply measures resistance using Ohms law at a point in time and does not allow information on electrode capacitance to be assessed. ECG electrodes are not concerned with dissipation of current over an area, only with the contact resistance that is created.

Therefore, there is a need for improved means to detect electrode peeling from the skin.

It is an object of the invention to obviate or mitigate the above drawbacks.

### SUMMARY OF THE INVENTION

The capacitance of the electrode to skin interface depends on the area of electrode contact and so a measurement of capacitance contains additional information about contact area. Prior art solutions focus only on the resistive component of electrode impedance and may be less effective in assessing area of contact. The equivalent circuit of the skin is known to be nonlinear and therefore linear approaches to measurement cannot always be relied upon.

According to a first aspect of the invention there is provided a system for assessing the quality of electrical contact in transcutaneous electrical stimulation, the system comprising: an array comprising at least two electrodes; control means for controlling flow of current pulses within electrode pairings of the array; measuring means for measuring at least one voltage sample between electrodes at at least one time point within a stimulation pulse; means for calculating a time dependent factor based on the voltage sample or samples; assessing means for assessing the quality of electrode contact; the assessing means configured to: compare the calculated time dependent factor with a pre-determined acceptance limit; characterise the quality of electrode contact as acceptable if the calculated time dependent factor is less than or equal to the pre-determined acceptance limit; and characterise the quality of electrode contact as unacceptable if the calculated time dependent factor is greater than the predetermined acceptance limit.

In one or more embodiments, a time dependent factor of voltage is a calculation that takes as its input the magnitude of a sample or samples of a voltage across a pair of electrodes as well as the time within the pulse at which the sample or samples were taken. Examples could include the rate of change of voltage between two time points, the difference between each sample and an upper limit which itself varies with time, a time constant of the voltage waveform, a cross correlation function with a template waveform, a coefficient of a polynomial curve fit of the voltage waveform. The time dependent factor may also be adjusted by the magnitude of the current used in the pulse in order to calculate a further factor, for example to estimate the capacitance. Alternatively, the magnitude of the current can be used to adjust the acceptance limit.

The measuring means may be configured to measure at least one voltage sample between electrodes at a plurality of time-points within the stimulation pulse.

In one or more embodiments, the time dependent factor is the difference between an initial voltage step and a voltage at a later time point in the stimulation pulse.

In one or more embodiments, the time dependent factor is the voltage at a defined later time point in the stimulation pulse.

In one or more embodiments, the time dependent factor is the difference between the initial voltage step and the voltage at the end of the pulse.

In one or more embodiments, the time dependent factor is an estimated time constant of a voltage waveform.

In one or more embodiments, the time dependent factor is an estimated rate of change of voltage with respect to time at a given time point.

In one or more embodiments, the time dependent factor is the electrode capacitance which is calculated by dividing an accumulated charge at a time point by a differential voltage at the time point.

In one or more embodiments, the time dependent factor is a coefficient of a polynomial model.

In one or more embodiments, the predetermined acceptance limit is dependent upon the magnitude of a current selected for the stimulation pulse.

In one or more embodiments, the acceptance limit is a maximum expected voltage value for the time point at a selected current within the stimulation pulse.

In one or more embodiments, the array comprises at least three electrodes and wherein the control means is configured to: drive a constant current between two of the at least three electrodes while sampling the voltage across these two electrodes and also at a third electrode, calculate the time dependent factor for the two electrodes and the third electrode, calculate a ratio of the time dependent factors and compare the ratio with an acceptance limit.

In one or more embodiments, the assessing means is configured to calculate the capacitance for each of the electrodes and to identify an electrode with the lowest capacitance as faulty.

In one or more embodiments, the array comprises at least three electrodes, wherein at least two electrode pairings of the array have a common electrode.

In one or more embodiments, the measuring means is configured to measure a plurality of voltages across each of the at least two electrode pairings of the array at a plurality of time points during the stimulation pulse.

In one or more embodiments, the measuring means is configured to measure voltages across each of three electrode pairings of the array at the plurality of time points during the stimulation pulse.

In one or more embodiments, the system comprises identifying means for identifying at least one faulty electrode by comparing measured voltages across each of the at three electrode pairings with at least one reference value in order to identify a faulty electrode.

In one or more embodiments, the assessing means is configured to identify at least one faulty electrode by calculating a voltage drop across at least one electrode and comparing the voltage drop to a predetermined acceptance limit in order to identify a faulty electrode.

In one or more embodiments, the system comprises an alerting means for alerting a user if one or more measured voltages exceed a reference value or a predetermined acceptance limit.

In one or more embodiments, the system further comprises a constant current controlled pulse generator for generating pulses of predetermined amplitude, duration and frequency.

In one or more embodiments, the system further comprises a bridge circuit for energising the electrodes, wherein the bridge circuit comprises a set of high side and low side switches for selecting electrodes to form a circuit.

In one or more embodiments, the system is a garment or belt based stimulation system.

In one or more embodiments, the array comprising the at least three electrodes is integrated into at least one of: a module, an applicator, a belt, or, a garment.

In second aspect of the present invention, there is provided a method of assessing the quality of electrical contact in transcutaneous electrical stimulation, the method comprising: forming an array comprising at least two electrodes; controlling flow of current pulses within electrode pairings of the array; measuring at least one voltage sample between electrodes at at least one time point within a stimulation pulse; calculating a time dependent factor based on the voltage sample or samples; and assessing the quality of electrode contact by: comparing the calculated time dependent factor with a pre-determined acceptance limit; characterising the quality of electrode contact as acceptable if the calculated time dependent factor is less than or equal to the pre-determined acceptance limit; and characterising the quality of electrode contact as unacceptable if the calculated time dependent factor is greater than the predetermined acceptance limit.

According to a further aspect of the present invention, there is provided a system for assessing the quality of electrical contact in transcutaneous electrical stimulation, the system comprising: an array comprising at least three electrodes, wherein at least two electrode pairings of the array have a common electrode; control means for controlling flow of current pulses between different electrode pairings of the array; and measuring means for measuring at least one differential voltage across each of the at least two electrode pairings of the array during a stimulation pulse in response to a constant current pulse. The differential voltage is the difference between voltage at the start of the pulse and the voltage at a later time in the pulse.

The measuring means may be configured to measure a plurality of voltages across each of the at least two electrode pairings of the array at a plurality of time-points during the stimulation pulse in response to the constant current pulse.

The system may further comprise identifying means for identifying at least one faulty electrode by comparing at least one measured differential voltage across each of the at least two electrode pairings (AB, BC) with reference values.

The measuring means may be configured to measure differential voltages across each of three electrode pairings of the array at the plurality of time-points during the stimulation pulse in response to the constant current pulse.

The system may further comprise identifying means for identifying at least one faulty electrode by comparing measured differential voltages across each of the at three electrode pairings (AB, AC, BC) with reference values.

The identifying means may be configured to identify the at least one faulty electrode by calculating a voltage drop at each of the three electrodes (A, B, C) and comparing the voltage drop to a predetermined acceptance limit.

The system may further comprise alerting means for alerting a user if one or more measured voltages exceed a reference value or a predetermined acceptance limit.

The system may further comprise a constant current controlled pulse generator for generating pulses of predetermined amplitude, duration and frequency, typically in the range 0 to 150 mA.

The system may further comprise a bridge circuit for energising the at least three electrodes, wherein the bridge circuit may comprise a set of high side and low side switches for selecting electrodes to form a circuit.

The system may be a garment or belt based stimulation system.

The array comprising the at least three electrodes (A, B, C) may be integrated into at least one of: a module, an applicator, a belt, or, a garment.

In a still further aspect of the present invention, there is provided a method of assessing the quality of electrical contact in transcutaneous electrical stimulation, the method comprising: forming at least two electrode pairings from an array comprising at least three electrodes, wherein the at least two electrode pairings of the array have a common electrode; controlling flow of current pulses between different electrode pairings of the array; and measuring at least one voltage across each of the at least two electrode pairings of the array during a stimulation pulse in response to a constant current pulse.

All essential, preferred or optional features or steps of one of the first aspect of the invention can be provided in conjunction with the features of the second aspect of the invention and vice versa.

### BRIEF DESCRIPTION OF THE DRAWINGS

Embodiments of the present invention are described hereinafter with reference to the accompanying drawings in which:

FIG. 1 shows a circuit model of transcutaneous stimulation;

FIG. 2 shows a circuit model with two electrodes in series;

FIG. 3 shows a circuit model with a three-electrode configuration;

FIG. 4 shows a current and voltage relationship for a constant current square wave through a pair of transcutaneous electrodes;

FIG. 5 shows an example implementation of the system of the present invention, including means for measuring electrode voltages during a stimulation current pulse i. and skin contact electrodes A, B and C;

FIG. 6 shows three electrodes, e1, e2 and e3 positioned on the abdomen of a person;

FIG. 7 shows a predicted voltage due to current of 10 mA in the model of FIG. 1;

FIG. 8 shows the voltage across a pair of electrodes for 6 users, current 24 mA, 290 μs pulse. Vertical axis in Volts;

FIG. 9 shows the voltage waveform during a single pulse of 300 μs for a range of current levels in a single user;

FIG. 10 shows voltage (ADC count) with respect to time across two electrodes in a series circuit and at a third monitoring electrode. The ratio of the voltages with time is also plotted;

FIG. 11 shows the differential voltage V-V1 for both the series connected electrodes and the third electrode for electrodes of equal contact area. Note the ratio throughout is approximately 50%;

FIG. 12 shows the differential voltage V-V1 for both the series connected electrodes and the third electrode for electrodes with mismatched contact areas. Note the clear mismatch in electrode voltages where the ratio is changed to less than 40%;

FIG. 13 shows a comparison of electrode voltage waveforms for the same current pulse with different electrolytes; Chi current (35 mA) Ch2 electrode voltage Hydrogel Sheet;

FIG. 14 shows an MCU based stimulation controller, including a constant current control and an output switch array.

### DETAILED DESCRIPTION OF THE INVENTION

Model of Skin Impedance

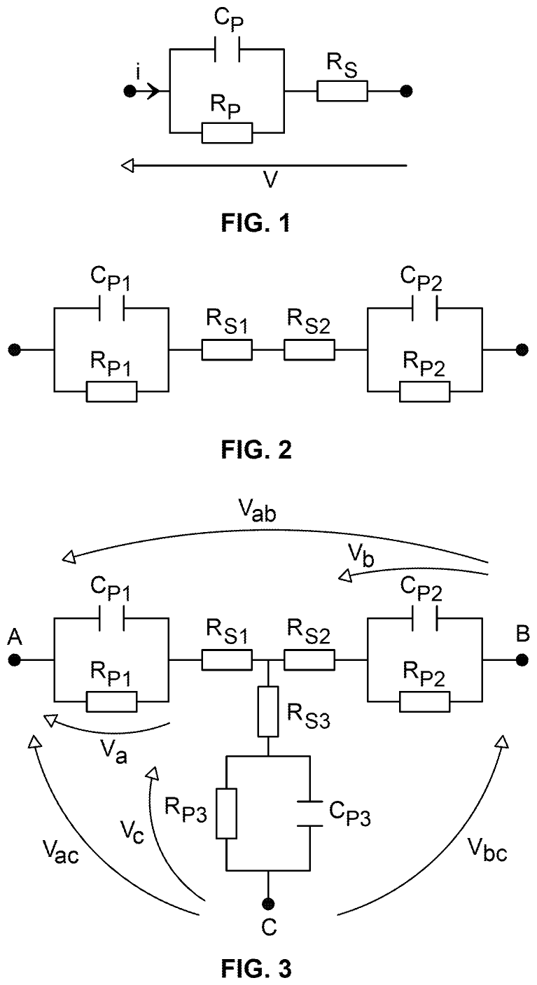

A widely used circuit model of transcutaneous stimulation is shown in FIG. 1. The parallel combination Rp and Cp represents the impedance of the stratum corneum (SC). This is the outer layer of the epidermis and it is composed of a lipid lamellae-corneocyte matrix arranged in bilayers (between 25 and 100) and has an approximate thickness between 10 to 100 μm. The layer has a relatively high electrical impedance but is traversed by appendages such as sweat glands and hair follicles which provide a lower impedance path for ion flow. Transfer of the current into the skin can occur by capacitive coupling across the stratum corneum, and this pathway is represented by Cp. Apart from the capacitive coupling, it is believed that there are two principal pathways for the current to cross the skin; the first being via the appendages and the other being through the corneocyte matrix. The resistive component Rp represents the electroporation that occurs when an electric field is applied across a membrane. Rp is known to be a nonlinear element, its value reducing as the current density increases and furthermore it depends on the accumulated charge transferred within a pulse and so is time dependent. Rp is considered ohmic for lower current densities and shorter pulses. The capacitor Cp is due to the charge storage which occurs across the thin layer of the SC. The Resistor Rs represents the resistance of tissue beneath the skin, in addition to the resistance of the leadwires from the stimulator to the electrodes. Rs is generally much lower than Rp.

It can be beneficial to assess additional time dependent aspects of the voltage waveform in order to assess the quality of skin contact. Considering the equivalent circuit of FIG. 1, the voltage in response to a current pulse of amplitude i is

$$V = {iR}_{s} + {iR}_{p} ( 1 - {e}^{- t / {CR}_{p}} ) \qquad equation 1$$

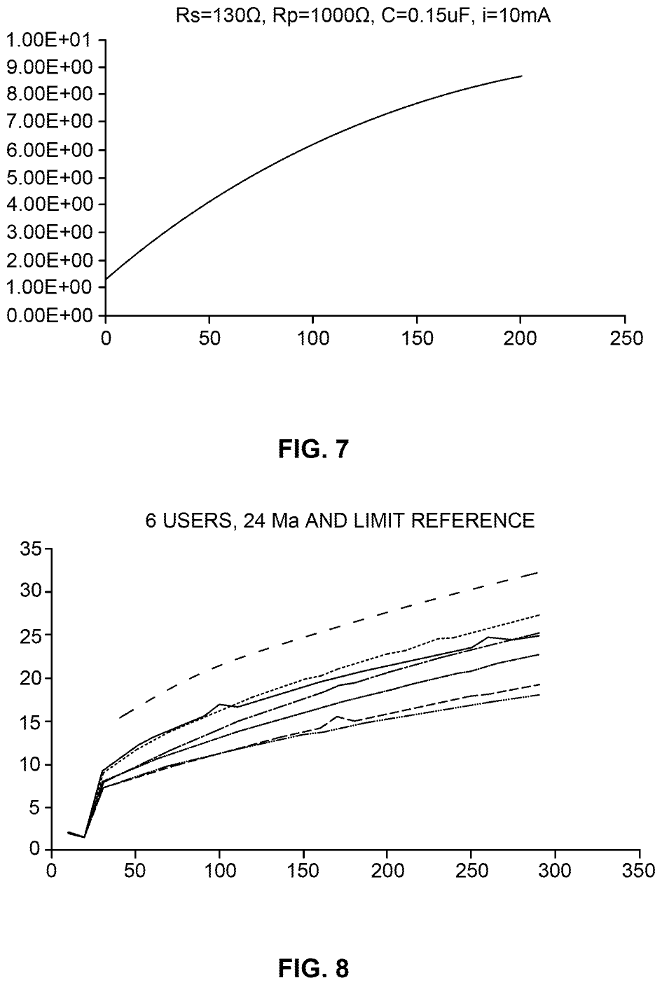

This has an initial step in voltage at the beginning corresponding to the first term above and then an inverse exponential component as the capacitance of the skin charges up. See FIG. 7 below.

If the time constant CRp is much greater than the pulse duration then the voltage waveform during the pulse will be approximately linear with respect to time. If not, then the waveform will follow a curve have a decreasing slope as the pulse progresses.

A simple quotient of voltage divided by current at the start or at an arbitrary time point in the pulse may not be sufficient to characterise the quality of electrode contact. The shape of the voltage waveform contains information about the capacitance of the skin-electrode contact and the resistance of the shunting parallel pathways. It is therefore preferable to sample at a plurality of time points during a stimulation pulse and to use the information so collected to gain information about the waveform shape and to classify the detected information as acceptable or not. For example, the voltage at a defined timepoint within the pulse is compared with a reference voltage limit for that time point. If it exceeds the limit it is classified as unacceptable, if less than or equal to the limit it is acceptable. A series of time dependent reference voltage limits can be derived from empirical data collected for the size of electrode in use, across a range of current levels.

FIG. 2 shows the equivalent circuit of two electrodes in series, although normally the two networks representing each of the electrodes are lumped into a single network of the same format, with suitably adjusted component values. FIG. 3 shows a three-electrode configuration.

Voltage and Current Relationship

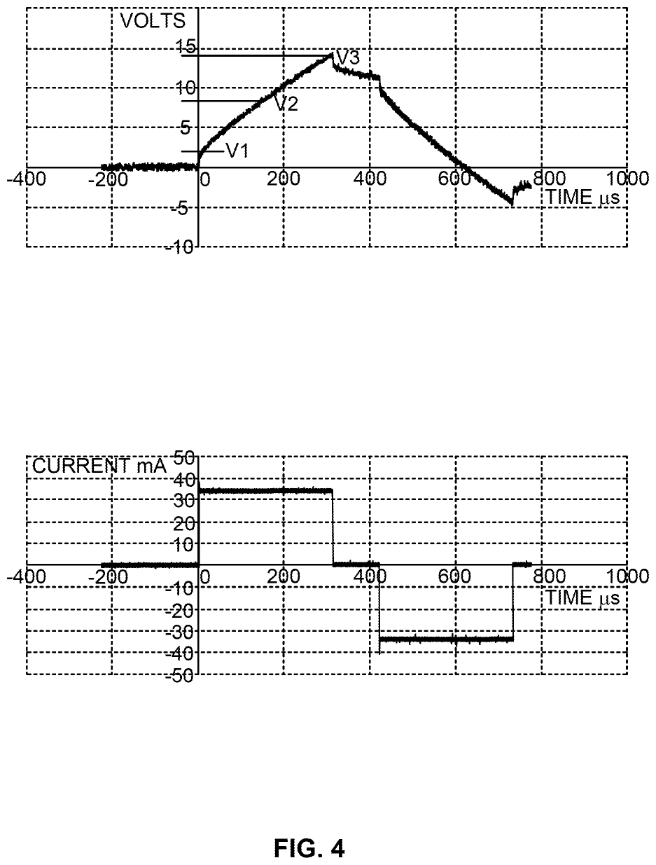

A typical current and voltage relationship for a constant current square wave through a pair of transcutaneous electrodes is shown in FIG. 4. The value of Rₛ is easily calculated from the initial step that occurs in the voltage waveform. Rₛ=V1/i, where V1 is the amplitude of the voltage step at the start of the pulse and i is the amplitude of the input current. We define the differential voltage at a time t within a pulse as the difference between the voltage at time t and the initial step voltage v1, that is,

V′=v(t)−V1

The ramp up of voltage during the pulse is due to the charge of Cp with some shunting of the current through Rp. Assuming that all the circuit resistances were ohmic, the capacitor would finally stop charging when the shunt current through Rp equalled the input current.

However, since Rp is nonlinear, it shunts a greater proportion of current as the current through it increases and as the pulse continues, thus leading to an earlier saturation of the voltage.

There is a voltage dependent effect related to electroporation. The permeability of a lipid membrane to ions is increased due to the application of an electric field. For the appendage pathway across the SC there are only a few layers involved and therefore the voltage required is low. For the corneocyte matrix of the SC however, there are many more layers (25 to 100) and the voltage required for electroporation can be much higher (>30V). We believe that electroporation at higher voltages can lead to skin irritation and should therefore be avoided. Accordingly, the detection of high voltage on the skin during a current pulse provides a means to detect and avoid this risk.

Description of One or More Embodiments of the Invention

The capacitance Cp depends on the area of the electrode making contact with the skin, assuming that the thickness of the dielectric provided by the SC remains unchanged. This capacitance charges during the stimulation pulse and the shape of the resultant voltage waveform can be used to compare between electrodes of an array and with reference values.

There is a trend towards using very large electrodes, having a contact area of greater than 50 cm², sometimes even reaching 600 cm² for single electrode. The capacitance of such electrodes can be very high and consequently the rate of change of voltage dv/dt is much lower than with smaller electrodes.

In one embodiment of the invention there is provided an apparatus for directing a pulse of current of known amplitude and duration to flow in series between two skin contacting electrodes and for sampling the resultant voltage across the electrodes at a plurality of time points in order to gain information about the waveform shape.

Consider a body worn garment or applicator containing at least two skin contacting electrodes of known area, where a 24 mA constant current pulse of duration 290 μs is applied. The resultant voltage waveform is sampled by an analog to digital converter every 10 us during the pulse, resulting in 30 measurements which could be indexed V1 through V30. A corresponding set of voltage limits is provided for a test current of 24 mA into a pair of electrodes and these are indexed R1 through R30. The test to be applied by the MCU is whether, for n=1 to 31 that Vn>Rn. It is not necessary to test each and every sample, it may be sufficient to use a subset of the available samples. See FIG. 8 which shows a sample voltage waveform collected from 6 subjects with normal electrodes and a linear time dependent voltage limit depicted as a dotted line.

In this way the system can determine whether the voltage waveform falls within an acceptable shape envelope at one or more specific time points.

The pulse can be terminated by the MCU at any sample point where the voltage exceeds the time dependent reference limit for that timepoint, or where more than a predetermined number of samples exceed their corresponding reference points. For example, a test could be simply a comparison of the measured voltage at a single timepoint of 100 μs, which in the above case is approximately 21V.

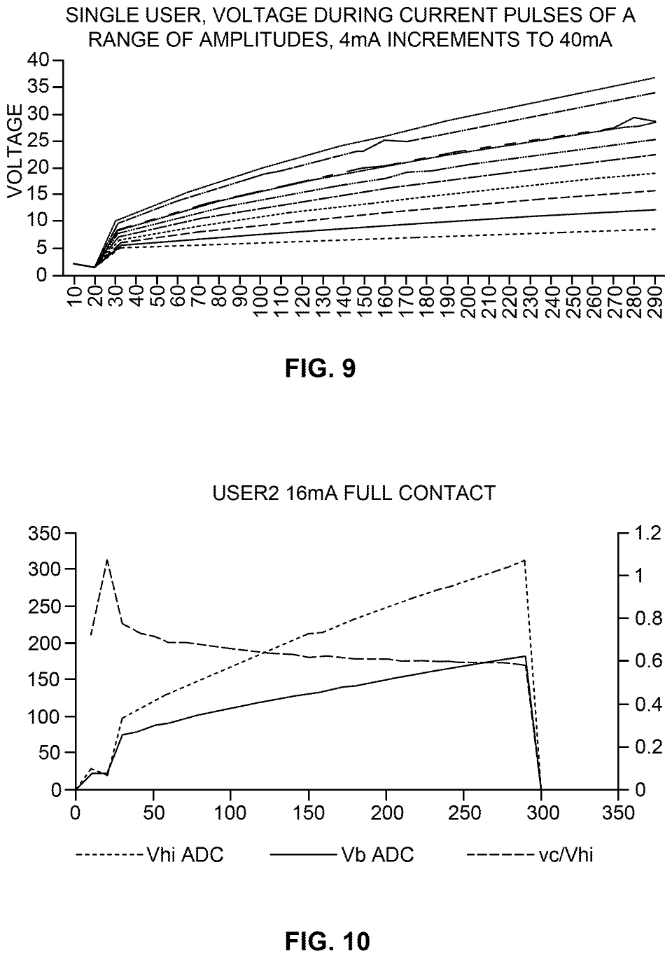

The set of reference limits can be extended to a longer pulse duration, or to higher amplitudes, or to different size or type of electrodes by empirical testing or analytic methods or a combination of these approaches. See FIG. 9 for an example of a series of voltage waveforms in a subject for a range of currents to 40 mA.

In certain conditions the capacitance of the electrodes can be estimated from the slope of the voltage waveform. The second term on the right hand side of equation 1 above can expanded in a power series expansion which gives

$$V = {iR}_{s} + i {R}_{p} 〈 \frac{t}{C {R}_{p}} - \frac{{t}^{2}}{2 {( C {R}_{p} )}^{2}} + \frac{{t}^{3}}{6 {( C {R}_{p} )}^{3}} )$$

where t is the time since the start of the pulse. When the time constant CRp is much greater than the pulse duration then the second and third order terms of the expansion can be ignored. If the initial voltage step is also subtracted from the entire waveform then we have

$$V - {V}_{1} = i ( \frac{t}{C} )$$

Therefore, the voltage at any point later in the pulse can be used to estimate the value of the capacitance.

The capacitance can be estimated with the following simple formula

$$C = i ( \frac{t}{V - {V}_{1}} )$$

The capacitance can therefore be estimated by calculating the charge delivered into the electrodes at a given timepoint into the pulse and dividing by difference between the voltage at that point and the initial step voltage v₁. The resultant estimate can be compared against reference values for the electrode concerned, which are stored in memory.

Estimating the slope dv/dt of each of these waveforms in FIG. 9 as V30-V1, allows us to estimate the capacitance as follows

| | | | | | | | | | | |
|---|---|---|---|---|---|---|---|---|---|---|
| I (mA) | 4.0 | 8.0 | 12.0 | 16.0 | 20.0 | 24.0 | 28.0 | 32.0 | 36.0 | 40.0 |
| dv/dt | 0.1 | 0.3 | 0.4 | 0.5 | 0.6 | 0.6 | 0.7 | 0.7 | 0.9 | 1.0 |
| V/μs | | | | | | | | | | |
| c = | 0.3 | 0.3 | 0.3 | 0.3 | 0.4 | 0.4 | 0.4 | 0.4 | 0.4 | 0.4 |
| i/(dv/dt) | | | | | | | | | | |
| (μF) | | | | | | | | | | |

Note that the estimate of capacitance is reasonably consistent across the range of currents. In this case a capacitance limit of 0.2 μF would be an appropriate reference limit for this electrode/electrolyte system. If the estimated capacitance was less than this limit a fault is signalled.

Where the time constant CRp is not much greater than the duration of the pulse then the second and third terms cannot be ignored and may be identified by curve fitting.

The time constant itself may be used as the factor to determine electrode quality of contact.

An important aspect of this invention is that some parameter related to electrode characteristics is available as a reference limit to the microcontroller. This can only be relied upon where the electrodes cannot be altered, replaced or modified or connected in a different way with respect to other electrodes. It is therefore more suitable for garments which integrate electrodes, wiring and connections in such a way that the user does not have to make any such adjustments.

A sample population of users can be assessed to measure the actual voltage waveform in response to a series of pulses across of range of current amplitudes. A statistical model is built of the mean and 95% confidence interval for upper end of the expected voltage envelope for a given current. An offset to the upper limit of the confidence interval can be applied to create a reference limit which is then used as the acceptance threshold for voltage at any point in the waveform.

Alternatively, an analytical model, such as that shown in equation 1 above, can be used to calculate the expected voltage in response to a given current at any point in the waveform. The model values Rp and C can be derived by experiment while Rs can be calculated directly from the amplitude of the initial step value. An offset can be applied to the calculated value to allow for normal variance in measurement and this value can be used as the acceptance threshold for voltage at any point in the waveform.

In the forgoing example the voltage waveform was sampled at successive timepoints during a pulse and compared with a time dependent reference limit. In a further example the slope of the waveform was used to estimate capacitance which can be used to compare to a reference limit. More generally, the sampled waveform data, or part of it, is used to estimate a time dependent voltage factor such as a slope, a time constant or other time dependent curve fit characteristic. A cross correlation of the sampled data with an ideal waveform shape template could be a further time dependent factor. The factor estimate can then be compared with an experimentally derived acceptance range of the factor for the electrode design in question.

For example, the voltage waveform slope towards the beginning of the pulse, due to a known current, could be estimated simply by subtracting sample 3 from sample 5 and dividing by the time difference in microseconds, e.g. 20 μs. The resultant value is compared with a table which gives an upper and lower limit for the parameter at this time point in the waveform. This could be useful for situations where the time constant CRp is less than the pulse duration. The slope could be estimated at other points in the waveform and controller could be programmed to terminate the pulse if the measured slope exceeds the reference limit for that time point. For other current values an adjustment factor can be provided which is experimentally derived for electrodes of the same size. A further adjustment factor can also be provided reflecting the fact that impedance can change with time during electrical stimulation. Various other factors could equally be derived from the set of samples, for example a time constant or coefficients of a 2^{nd} or 3^{rd} order polynomial fit. A selection of these factors can be compared with reference values to determine electrode acceptance. Since the skin-electrode model is known to be nonlinear, equation A1 cannot always be relied upon in the assessment of waveform shape, especially where peak current densities greater than 2 mA/cm² are involved. The empirical data defining the acceptance range of the voltage envelope during pulses of known amplitude is preferred in such situations.

In a further embodiment, there is provided an apparatus for assessing the quality of electrical contact in transcutaneous electrical stimulation comprising an array of at least three electrodes and a control means whereby current pulses of known amplitude and duration can be directed to flow between a first and second electrode while simultaneously monitoring the resultant voltage across the first and second electrode electrodes as well as a third electrode at a plurality of time points in order to gain information about the waveform shape at the first and second electrodes.

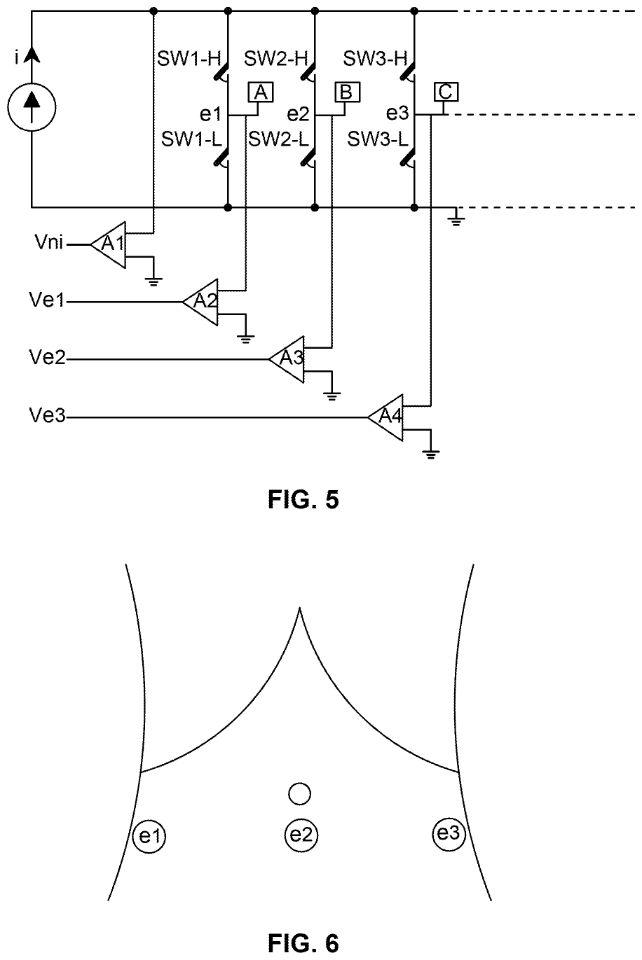

In a system of three (or more) electrodes as depicted in FIG. 3, where a current is directed between two of the electrodes, the observed voltage on a third electrode can be used to estimate the voltage across each of the two electrodes. The circuit for this is shown in FIG. 5 where each of the blocks A represents an attenuator which presents the measured voltage to an A to D converter port of the MCU. The MCU can sample the voltage on both Vₕᵢ and any one of Vₑ₁, Vₑ₂, Vₑ₃ etc, which are the skin contact electrode terminals. In the example of FIG. 3, the signal Vₕᵢ could give the voltage across the two electrodes A and B in series, while Vₑ₃ gives the voltage of the body, via electrode C. Assume that electrode A is the anode and electrode B is the cathode during the current pulse, then Vₑ₃ provides an estimate of the voltage V_{bc}. See FIG. 10 below in which the initial step in voltage can be seen, followed by the slower rise in voltage corresponding to the charging of Cₚ The charge on the capacitor can be estimated by simply subtracting the voltage step V1 as before.

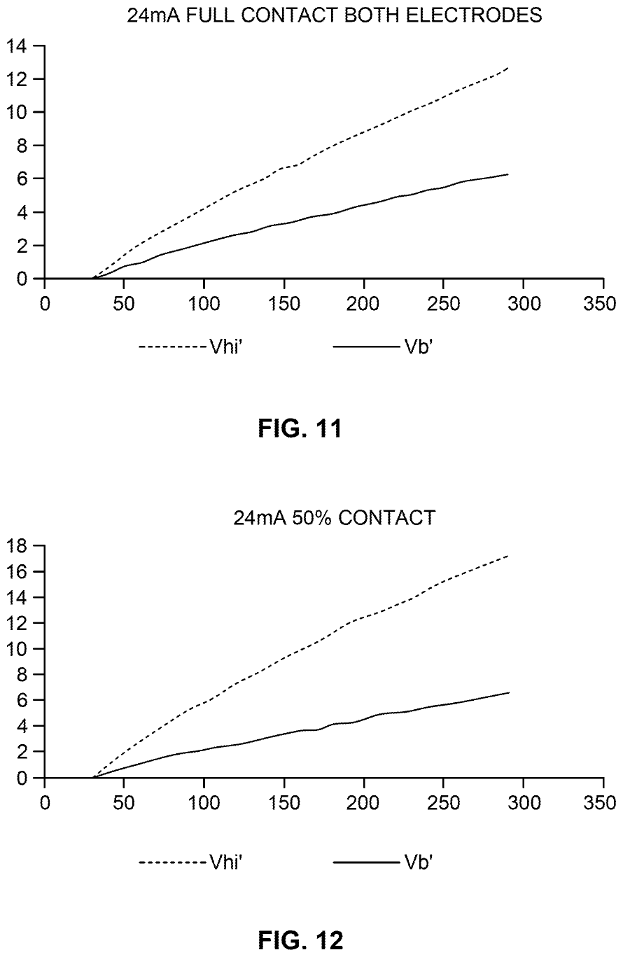

For two series connected electrodes of equal size where Rₛ₁=Rₛ₂, Rₚ₁=Rₚ₂, and C₁=C₂ then the voltage across each electrode skin interface is the same. See FIG. 11. If, however one electrode is partially peeled such that, for example C₂ is decreased by 50% while Rₚ₂ increases by 50%, then, since the same current passes through each electrode, the voltage accumulated across the lower value capacitance is greater. Therefore, the voltage difference between the electrodes increases with time and this fact can be used to detect electrode peeling.

See for example FIG. 12 which shows Vhi and Vb as before but where electrode A only makes 50% area of contact. The offset voltage due to Rs has been subtracted in each case to give only the voltage on the CRp part of the model. It can be seen that the voltages on the electrodes diverge as the pulse progresses. Therefore it is possible to detect electrode peeling by
(i) measuring the voltage waveform across two series connected electrodes due to a known current
(ii) simultaneously measuring the voltage at a third electrode and designating it the voltage across the cathode electrode.
(iii) Subtracting the respective step voltage V1 in each case (i) and (ii) to obtain only the capacitance voltage
(iv) Forming the ratio between the two estimates of capacitance voltage (ii)/(i)
(v) Comparing the ratio with an acceptance limit window
(vi) Signalling an error condition if outside the acceptance limit
(vii) Identifying which electrode has the highest voltage and signalling the identity of the electrode.

For example, if the two electrodes are intended to be the same size, then the acceptance window could be set between 40% and 60%. Any ratio outside that limit suggests a peeling electrode.

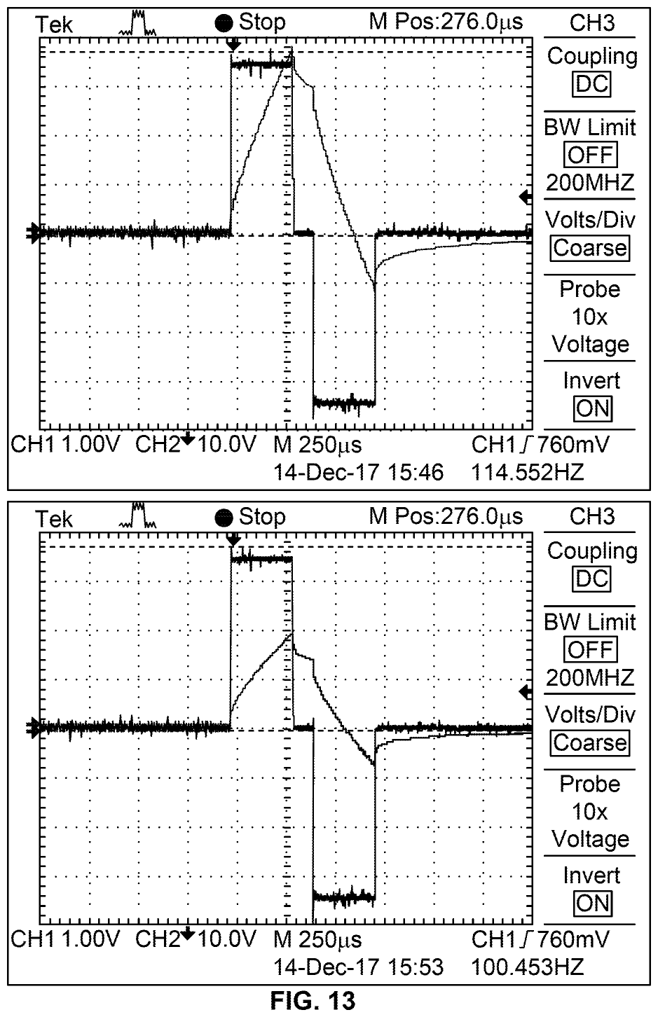

As well as having a range of sizes, transcutaneous electrodes can be implemented in several technologies that affect their impedance characteristics. Hydrogel sheets are often used as an electrolyte layer applied to a conductive layer which can be made of carbon film, conductive TPU, printed silver ink, conductive textile or metal foil. FIG. 13 shows a comparison of the electrode voltage for the same current between two types of electrolyte; a hydrogel layer and a saline spray. The electrode material, skin location and leadwires were otherwise the same. Note the voltage for the saline is much less than for the hydrogel. It is therefore necessary to define

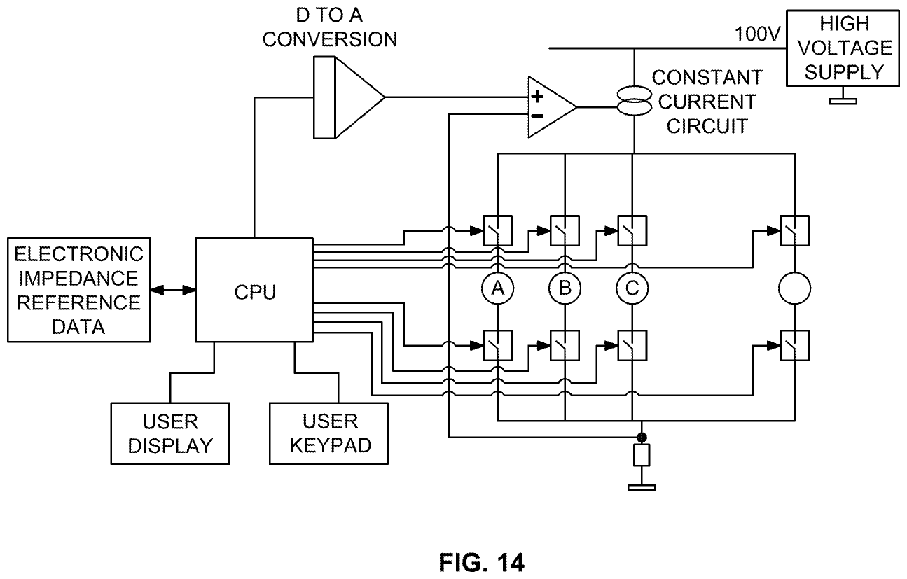

In the forgoing discussion it is assumed that the stimulator comprises an electronic module which contains a micro computer (MCU) or central processing unit (CPU) which is programmed to manage user input, waveform synthesis, waveform sampling, user display and communications. A typical block diagram for such a device is shown in FIG. 14 which also shows the current control circuit and output H-bridge array which is used to steer the current pulse to selected electrodes. The voltage monitoring depicted in FIG. 5 is not shown in this figure.

In one embodiment, there is provided an apparatus for assessing the quality of electrical contact in transcutaneous electrical stimulation comprising an array of at least three electrodes and a control means whereby current pulses can be directed to flow between different electrode pairs of the array and wherein at least two pairs have one common electrode.

A means of measuring the voltage at one or more time-points during a stimulation pulse is provided in response to a constant current pulse, for example, a first voltage V1 being the amplitude of the step voltage at the start of the pulse and a second voltage V2 being the voltage mid-way through the pulse and a third voltage V3 being the amplitude of the pulse at the end of the phase.

Consider three electrodes labelled A, B and C as illustrated in FIG. 3. There are three possible electrode pairings [A,B], [A,C] and [B,C]. A current pathway can be established in any of these pairs, where the unused electrode is unconnected. Consider two pairs [A,B] and [B,C] from the array. First, a current pulse of known amplitude i1 is applied across pair [A,B] and voltages samples V1 and V3 are recorded at the beginning and end of the phase. Since we are concerned with the quality of the skin contact and not the resistance of the subcutaneous tissues we subtract V1 from V3 to get the accumulated voltage drop across the skin

Vab′=V3−V1

This is labelled Vab′ to associate it with the pair [A,B] and to denote that it refers to the sum of the voltage drops across the two electrodes of the pair. Next, we apply the same amplitude and duration current pulse to pair [B,C] and measure at the same timepoints to obtain Vbc′

Referring to FIG. 3 we can see that, voltage drop across Rsi

Vab′=Va+Vb and Vbc′=Vb+Vc

So that Vab′−Vbc′=Va−Vc.

Since the intended area of skin contact of each of the electrodes is known in advance, an approximate expected value for each of Vab′ and Vbc′, as well as their difference, can be defined. By comparing the measured values with these predetermined limits the location of the high resistance electrode can be identified. If both Vab′ and Vbc′ exceed the limit it is likely that the common electrode B is at fault. If either one of Vab′ or Vbc′ are within the limit then the faulty electrode is likely to be the opposite one.

By measuring a third pair [A,C] it is possible to estimate the voltage across each electrode, as distinct from across a series connected pair of electrodes, since now we have a complete set of simultaneous equations. Assume the corresponding voltages for pair [A,C] are available

| | |
|---|---|
| Vab′ = Va + Vb | 1 |
| Vbc′ = Vb + Vc | 2 |
| Vac′ = Va + Vc | 3 |
| Vab′ − Vbc′ = Va − Vc | 4 |
| Vab′ − Vac′ = Vb − Vc | 5 |
| Vbc′ − Vac′ = Vb − Va | 6 |

Adding equations 4 and 5 and using equation 1 to eliminate Va+Vb we get

Vc=(Vac+Vbc−Vac)/2

Similar equations can be arrived at for Vb and Vc.

Va=(Vab+Vac−Vbc)/2

Vb=(Vab+Vbc−Vac)/2

In this way an estimate of the voltage drop at each electrode can be made and compared with an acceptance limit. This allows the faulty electrode to be identified which, when communicated to the user, is of great benefit in remedying the fault. For an array of electrodes of known design and area of contact the expected values of the electrode voltages can be determined by analysis and or experiment. This can include arrays of electrodes where not all of the electrodes are the same size. To allow for normal variation in electrode impedance, for example due to skin type, a normal acceptance range of voltages can be developed. An abnormal condition can therefore be detected when a voltage is measured outside the predefined normal range. It is known that electrode impedance can change during the course of a stimulation session, however the relative impedance of one electrode compared to another should not change significantly. Therefore, limits of acceptable difference between electrode voltages can be defined and the user alerted when an electrode falls outside the acceptance range.

While the foregoing discussion related to 3 electrodes, it is readily seen that the principle is valid for any number of electrodes greater than 3, where electrodes can be selected in pairs.

It is also clear that the principle embodied in this invention can be applied to voltage samples taken at other points in the waveform. The voltage sample V1 allows direct estimation of the series resistance Rs. Normally this represents the resistance of the subcutaneous tissues but its value would increase if another series resistance develops in the circuit. In the foregoing analysis it has been assumed Rs is lower than the skin resistance Rp. In modern garment based stimulation systems the conductors are often made from conductive thread or printed ink. These conductors, or the electrodes themselves, can develop high resistance resulting in reduced performance. The embodiment described here can be used to determine the series resistance of each branch of the model and alert the user accordingly. Preferably, the value of Rs in each branch is validated to be within an acceptable range before proceeding to evaluate further samples. If the value of Rs was found to be unacceptably high, then interpretation of subsequent assessments of electrode area of contact could be unreliable.

An example implementation of the forgoing embodiment is shown in FIG. 5. Three electrodes, e1, e2 and e3 are shown positioned on the abdomen (see FIG. 6) and these would normally be integrated into a belt or garment such that their size and relative position is fixed. A constant current controlled pulse generator is provided which can generate pulses of predetermined amplitude, duration and frequency, typically in the range 0 to 150 mA. Three electrodes are energised from a bridge circuit comprising a set of high side and low side switches which are under the control of a microprocessor (not shown here, but see FIG. 14). Terminal e1 connects to electrode e1 and so on for the other electrodes. To select electrodes e1 and e2 to form a series circuit and receive a pulse of current the corresponding high and low side switches are activated, S1h closed with S1l open for e1 to be the anode, while S2h open with S21 closed are selected for e2 to be the cathode. The voltage across the bridge at any instant, see V_{N} of FIG. 5, is in effect the voltage across the selected electrode pair (ignoring the voltage drops on the switches) and it can be measured through an attenuator A and analog to digital converter. The resulting digitised sample is read by the microcontroller software and processed according to an algorithm. Modern microcontrollers have built in analog to digital converters and moreover can sample at sufficient time and amplitude resolution. Direct memory access allows samples to be automatically streamed into memory. Such microcontrollers have sufficient speed to calculate differential voltages, to calculate capacitance, to compare calculated values with a lookup table of reference values.

The microcontroller selects pair e1 e2 and applies a pulse of known current amplitude. It reads V1 and V3 and stores them It calculates the differential V3-V1. After 10 milliseconds or longer, to ensure any body capacitance has discharged, it repeats the measurement and calculation on e1, e3. It waits a further period before testing the e2, e3 pair. When measuring on a third electrode, as illustrated in FIG. 14, an additional analog to digital converter can be used or a multiplexor to switch between 2 or more sources. The delay between samples is negligible in the present context.

A first step is to see if the series resistance Rs of each branch is within limits. This is done using the V1 sample for each branch which is compared with a predetermined limit stored in memory. If the measured voltage exceeds the limit for all three branches then at least two of the electrodes are faulty, but it may not be possible to identify which they are. If two of the branches exceed the limit while the third does not, then the problem is with the common electrode of the two branches. The processor then alerts the user by an audible or visible indicator (not shown).

Alternatively, or additionally, the processor may calculate the effective series resistance of each electrode by solving the set of simultaneous equations described above. At the very start of the pulse the capacitor in the skin model appears as a short circuit so the equivalent circuit of FIG. 3 reduces to the star connection of the Rs resistors. It is therefore critical to sample the voltage immediately after the pulses start to identify Rs.

Assuming the value of Rs is within limits the processor can now analyse the skin resistance part of the model. Subtracting V1 from V3 for each of the branches leaves the voltage across the skin only, that is, the series connection of two skin interfaces corresponding to the two electrodes of the pair. To maximise the signal to noise ratio it is important to sample the voltage waveform just before the end of the pulse, since at this point the voltage across the skin is at its maximum.

The microprocessor solves the set of simultaneous equations to find the voltage drop across the skin at each electrode. This is compared with a predetermined limit which is retrieved from memory. If the voltage exceeds the limit then the user is alerted and the faulty electrode is identified, for example on the on-screen diagram (not shown). The predetermined limits for electrode voltages can be developed by theory and or experiment. The voltage depends on the size, shape and relative location of the electrode, as well as its material construction. Since the electrodes are commonly built into garments their construction, size and relative position are fixed and so the predetermined limits remain valid. The variable aspects are the quality of the electrolyte, wear and tear of the garment, mis-application of the garment and the invention can help to detect these problems.

During the stimulation session the technique can be used to continuously monitor the electrodes, comparing them both with their baseline values and with each other. A marked increase in voltage relative to baseline and or another of the electrode suggests that the quality of the connection has deteriorated.

This technique allows the system to discriminate to some extent between a fault caused by the appearance of series resistance in the electrode and/or its lead-wire and a reduction in the area of contact of the electrode with the skin.

U.S. Pat. No. 6,728,577 B2 describes arrays of electrodes and switches to direct current in various pathways through the array. International patent application number PCT/IB02/03309 published under international publication number WO 03/006106 A2 contains more detail on switching and in FIG. 10 provides a block schematic of how the constant current control is setup with respect to the switching array. These two documents provide detail about the control means mentioned in the present invention with regard to electrode pair selection, current control and garment integration of electrodes. The disclosure of both of these documents is herein incorporated by reference.

In the foregoing description, unless otherwise specified the terms “electrode” and “electrodes” are used interchangeably.

Modifications are possible within the scope of the invention, the invention being defined in the appended claims.

### REFERENCES

Vargas Luna, J. L, M. Krenn, J. A. Cortes Ramirez and W. Mayr (2015). “Dynamic impedance model of the skin-electrode interface for transcutaneous electrical stimulation.” PLoS One 10(5): e0125609.

1. A system for assessing quality of electrical contact in transcutaneous electrical stimulation, the system comprising:
i. an array comprising at least two electrodes;
ii. a controller for controlling flow of current pulses within electrode pairings of the array; and
iii. a voltage measurement device for measuring at least one voltage sample between electrodes at at least one time point within a stimulation pulse;
iv. a calculator for calculating a time dependent factor based on the voltage sample or samples;
v. an assessor for assessing quality of electrode contact; the assessor configured to:
compare the calculated time dependent factor with a pre-determined acceptance limit;
characterise the quality of electrode contact as acceptable if the calculated time dependent factor is less than or equal to the pre-determined acceptance limit; and
characterise the quality of electrode contact as unacceptable if the calculated time dependent factor is greater than the predetermined acceptance limit.

2. The system of claim 1, wherein the voltage measurement device is configured to measure at least one voltage sample between electrodes at a plurality of time-points within the stimulation pulse.

3. The system of claim 1, wherein the time dependent factor is the difference between an initial voltage step and a voltage at a later time point in the stimulation pulse.

4. The system of claim 1, wherein the time dependent factor is the voltage at a defined later time point in the stimulation pulse.

5. The system of claim 3, wherein the time dependent factor is the difference between the initial voltage step and the voltage at the end of the pulse.

6. The system of claim 1, wherein the time dependent factor is an estimated time constant of a voltage waveform.

7. The system of claim 1, wherein the time dependent factor is an estimated rate of change of voltage with respect to time at a given time point.

8. The system of claim 1, wherein the time dependent factor is the electrode capacitance which is calculated by dividing an accumulated charge at a time point by a differential voltage at the time point.

9. The system of claim 1, wherein the time dependent factor is a coefficient of a polynomial model.

10. The system of claim 1, wherein the predetermined acceptance limit is dependent upon the magnitude of a current selected for the stimulation pulse.

11. The system of claim 1, wherein the acceptance limit is a maximum expected voltage value for the time point at a selected current within the stimulation pulse.

12. The system of any preceding claim 1, wherein the array comprises at least three electrodes and wherein the means controller is configured to: drive a constant current between two of the at least three electrodes while sampling the voltage across the two of the at least three electrodes and also at a third electrode, calculate the time dependent factor for the two of the at least three electrodes and the third electrode, calculate a ratio of the time dependent factors and compare the ratio with an acceptance limit.

13. The system of claim 12, wherein the assessor is configured to calculate the capacitance for each of the electrodes and to identify an electrode with the lowest capacitance as faulty.

14. The system of claim 1, wherein the array comprises at least three electrodes (A, B, C), wherein at least two electrode pairings (AB, BC) of the array have a common electrode (B).

15. The system of claim 14, wherein the voltage measurement device is configured to measure a plurality of voltages (V1, V2, V3) across each of the at least two electrode pairings (AB, BC) of the array at a plurality of time points during the stimulation pulse.

16. The system of claim 14, wherein the voltage measurement device is configured to measure voltages across each of three electrode pairings (AB, AC, BC) of the array at the plurality of time points during the stimulation pulse.

17. The system of claim 16, further comprising an identifier for identifying at least one faulty electrode by comparing measured voltages across each of the at three electrode pairings (AB, AC, BC) with at least one reference value in order to identify a faulty electrode.

18. The system of claim 17, wherein the assessor is configured to identify at least one faulty electrode by calculating a voltage drop across at least one electrode and comparing the voltage drop to a predetermined acceptance limit in order to identify a faulty electrode.

19. The system of claim 1, further comprising a signal for alerting a user if one or more measured voltages exceed a reference value or a predetermined acceptance limit.

20. The system of claim 1, further comprising a constant current controlled pulse generator for generating pulses of predetermined amplitude, duration and frequency.

21. The system of claim 1, further comprising a bridge circuit for energizing the electrodes, wherein the bridge circuit comprises a set of high side switches and low side switches for selecting electrodes to form a circuit.

22. The system of claim 1, wherein the system is a garment or belt based stimulation system.

23. The system of claim 1, wherein the array comprising the at least three electrodes (A, B, C) is integrated into at least one of: a module, an applicator, a belt, or, a garment.

24. A method of assessing quality of electrical contact in transcutaneous electrical stimulation, the method comprising:
i. forming an array comprising at least two electrodes;
ii. controlling flow of current pulses within electrode pairings of the array;
iii. measuring at least one voltage sample between electrodes at at least one time point within a stimulation pulse;
iv. calculating a time dependent factor based on the voltage sample or samples; and
v. assessing the quality of electrode contact by:
comparing the calculated time dependent factor with a pre-determined acceptance limit;
characterising the quality of electrode contact as acceptable if the calculated time dependent factor is less than or equal to the pre-determined acceptance limit; and
characterising the quality of electrode contact as unacceptable if the calculated time dependent factor is greater than the predetermined acceptance limit.
